(19) United States
(12) Patent Application Publication
(10) Pub. No.: US 2024/0328135 A1
(43) Pub. Date: Oct. 3, 2024

(54) TOILET DEVICE, METHOD FOR CONTROLLING TOILET DEVICE, AND RECORDING MEDIUM

(71) Applicant: TOTO LTD., Kitakyushu-shi (JP)

(72) Inventors: Yutaro TERADA, Kitakyushu-shi (JP); Satoshi KAWADA, Kitakyushu-shi (JP); Takamasa SUZUKI, Kitakyushu-shi (JP); Junki HAMADA, Kitakyushu-shi (JP)

(21) Appl. No.: 18/598,499

(22) Filed: Mar. 7, 2024

(30) Foreign Application Priority Data
Mar. 31, 2023 (JP) 2023-058670
Mar. 31, 2023 (JP) 2023-058672

(51) Int. Cl. E03D 9/00; G01N 33/493; H04W 4/30

(52) U.S. Cl. CPC E03D 9/00; G01N 33/493; H04W 4/30

(57) ABSTRACT

A toilet device includes a casing, a biological information measurement sensor, a controller, and a wireless communication part. The biological information measurement sensor is configured to measure biological information of a user. The controller is disposed inside the casing. The controller is configured to transmit the biological information to a personal digital assistant of the user. The wireless communication part is configured to connect the controller and the personal digital assistant by a wireless communication. The controller is connected with the personal digital assistant when a distance between the personal digital assistant and the wireless communication part is not more than a prescribed distance. The wireless communication part is disposed inside the casing.

### CROSS-REFERENCE TO RELATED APPLICATIONS

This application is based upon and claims the benefit of priority from Japanese Patent Application No. 2023-058672, filed on Mar. 31, 2023; and No. 2023-058670, filed on Mar. 31, 2023; the entire contents of which are incorporated herein by reference.

### FIELD

Embodiments described herein relate generally to a toilet device, a method for controlling a toilet device, and a recording medium.

### BACKGROUND

There is a biological information measurement system that performs wireless communication when a wearable authentication device and a personal authentication device approach each other within a prescribed distance (e.g., 2006-284232 JP A). In such a biological information measurement system, for example, wireless communication with a personal authentication device disposed at a wall surface of a toilet room is performed when a user wearing a wearable authentication device approaches.

When, however, the personal authentication device is disposed at the wall surface of the toilet room, the personal authentication device must be disposed at a position at which the personal authentication device be supplied with power and can communicate with a controller of a toilet bowl device; and the degree of freedom of the layout inside the toilet room may be reduced. Furthermore, when the personal authentication device is disposed at the wall surface of the toilet room, the authentication accuracy may be reduced because water easily contacts the personal authentication device.

### SUMMARY

According to the embodiment, a toilet device includes a casing, a biological information measurement sensor, a controller, and a wireless communication part. The biological information measurement sensor is configured to measure biological information of a user. The controller is disposed inside the casing. The controller is configured to transmit the biological information to a personal digital assistant of the user. The wireless communication part is configured to connect the controller and the personal digital assistant by a wireless communication. The controller is connected with the personal digital assistant when a distance between the personal digital assistant and the wireless communication part is not more than a prescribed distance. The wireless communication part is disposed inside the casing.

### BRIEF DESCRIPTION OF THE DRAWINGS

FIG. 1 is a perspective view illustrating a toilet system including a toilet device according to an embodiment;

FIG. 2 is a block diagram illustrating the toilet system including the toilet device according to the embodiment;

FIG. 3 is a front view illustrating a remote control of the toilet device according to the embodiment;

FIG. 4 is a flowchart illustrating an example of an operation of a registration mode of the toilet device and the personal digital assistant according to the embodiment;

FIG. 5 is a flowchart illustrating an example of an operation of a use mode of the toilet device and the personal digital assistant according to the embodiment;

FIG. 6 is a flowchart illustrating an example of an operation when connecting the toilet device and the personal digital assistant according to the embodiment;

FIG. 7 is a flowchart illustrating an example of an operation in the user authentication of the toilet device and the personal digital assistant according to the embodiment;

FIG. 8 is a flowchart illustrating an example of an operation in the user authentication by the remote control of the toilet device according to the embodiment;

FIG. 9 is a flowchart illustrating an example of an operation of the use mode of the toilet device according to the embodiment;

FIG. 10 is a flowchart illustrating an example of an operation in the user authentication of the toilet device according to the embodiment;

FIG. 11 is a flowchart illustrating an example of an operation in the user authentication of the toilet device and the personal digital assistant according to the embodiment;

FIG. 12 is a flowchart illustrating an example of an operation in the user authentication of the toilet device and the personal digital assistant according to the embodiment;

FIG. 13 is a plan view illustrating a part of the toilet device according to the embodiment;

FIG. 14 is a side cross-sectional view illustrating a part of the toilet device according to the embodiment;

FIG. 15 is a side cross-sectional view illustrating a part of the toilet device according to the embodiment; and

FIG. 16 is a plan view schematically illustrating the toilet device according to the embodiment.

### DETAILED DESCRIPTION

A first aspect is a toilet device including a casing, a biological information measurement sensor configured to measure biological information of a user, a controller that is disposed inside the casing and configured to transmit the biological information to a personal digital assistant of the user, and a wireless communication part configured to connect the controller and the personal digital assistant by a wireless communication; the controller is connected with the personal digital assistant when a distance between the personal digital assistant and the wireless communication part is not more than a prescribed distance; and the wireless communication part is disposed inside the casing.

According to the toilet device, water contact with the wireless communication part can be suppressed by disposing the wireless communication part inside the casing. Also, by disposing the wireless communication part inside the casing, for example, compared to when the wireless communication part is disposed at a wall surface of the toilet room, a reduction of the degree of freedom of the layout inside the toilet room can be suppressed.

A second aspect is the toilet device of the first aspect, wherein at least a part of the wireless communication part is positioned higher than a vertical-direction center of the casing, and whether or not the distance between the personal digital assistant and the wireless communication part is not more than the prescribed distance is determined by whether or not a strength of a radio signal emitted from the wireless communication part and received by the personal digital assistant is not less than a threshold.

According to the toilet device, whether or not the distance between the personal digital assistant and the wireless communication part is not more than the prescribed distance is determined by whether or not the strength of the radio signal emitted from the wireless communication part and received by the personal digital assistant is not less than the threshold; as a result, the strength of the radio signal received by the personal digital assistant can be used to easily determine whether or not the distance between the personal digital assistant and the wireless communication part is not more than the prescribed distance. The detection accuracy of the wireless communication part can be increased by disposing the wireless communication part so that at least a part of the wireless communication part is positioned higher than the vertical-direction center of the casing.

A third aspect is the toilet device of the second aspect, wherein at least a part of the wireless communication part is positioned behind a longitudinal-direction center of the casing.

According to the toilet device, the wireless communication part can be separated from the opening of the toilet seat by disposing the wireless communication part so that at least a part of the wireless communication part is positioned behind the longitudinal-direction center of the casing. As a result, for example, dropping the personal digital assistant through the opening of the toilet seat into the bowl of the toilet when passing the personal digital assistant over the wireless communication part to perform user authentication can be suppressed.

A fourth aspect is the toilet device of the second or third aspect, wherein the wireless communication part is positioned leftward of a lateral-direction center of the casing, or rightward of the lateral-direction center of the casing.

According to the toilet device, the wireless communication part can be separated from the opening of the toilet seat by disposing the wireless communication part to be positioned leftward of the lateral-direction center of the casing or rightward of the lateral-direction center of the casing. As a result, for example, dropping the personal digital assistant through the opening of the toilet seat into the bowl of the toilet when passing the personal digital assistant over the wireless communication part to perform user authentication can be suppressed.

A fifth aspect is the toilet device of the third aspect, wherein the casing includes a first upper surface part not overlapping the wireless communication part in the vertical direction, and a second upper surface part that is positioned behind the first upper surface part and overlaps the wireless communication part in the vertical direction; and an incline angle of the second upper surface part with respect to a horizontal plane is less than an incline angle of the first upper surface part with respect to the horizontal plane.

According to the toilet device, the second upper surface part can be a more horizontal surface by setting the incline angle with respect to the horizontal plane of the second upper surface part, which overlaps the wireless communication part in the vertical direction, to be less than the incline angle with respect to the horizontal plane of the first upper surface part, which does not overlap the wireless communication part in the vertical direction. As a result, for example, the personal digital assistant does not easily slide frontward when passing the personal digital assistant over the wireless communication part to perform the user authentication; and dropping the personal digital assistant through the opening of the toilet seat into the bowl of the toilet can be suppressed.

A sixth aspect is the toilet device of the second or third aspect, wherein the casing includes an indication part disposed at a position at which the indication part overlaps the wireless communication part in the vertical direction.

According to the toilet device, by disposing the indication part at a position at which the indication part overlaps the wireless communication part in the vertical direction, the user can more easily ascertain the position of the wireless communication part. As a result, for example, the user authentication is easily performed when the personal digital assistant is passed over the wireless communication part to perform the user authentication.

A seventh aspect is the toilet device of the first aspect, wherein the toilet device further includes a storage part; the controller is configured to perform a registration mode that includes associating identification information identifying the personal digital assistant and user information identifying the user, and storing the identification information and the user information in the storage part; after performing the registration mode, the controller performs a user authentication of designating the user information and the identification information when the distance between the personal digital assistant and the wireless communication part becomes the prescribed distance or less; and the prescribed distance is modifiable by the personal digital assistant.

According to the toilet device, by making the prescribed distance modifiable by the personal digital assistant, the distance (the prescribed distance) within which the user authentication is performed can be set to an appropriate distance regardless of the physique of the user and the posture when the user utilizes the toilet device. The user authentication can be more reliably performed thereby.

An eighth aspect is the toilet device of the seventh aspect, wherein whether or not the distance between the personal digital assistant and the wireless communication part is not more than the prescribed distance is determined by whether or not a strength of a radio signal emitted from the wireless communication part and received by the personal digital assistant is not less than a threshold; and the threshold is modifiable by the personal digital assistant.

According to the toilet device, whether or not the distance between the personal digital assistant and the wireless communication part is not more than the prescribed distance is determined by whether or not the strength of the radio signal emitted from the wireless communication part and received by the personal digital assistant is not less than the threshold; as a result, the strength of the radio signal received by the personal digital assistant can be used to easily determine whether or not the distance between the personal digital assistant and the wireless communication part is not more than the prescribed distance. On the other hand, when determining whether or not the strength of the radio signal emitted from the wireless communication part and received by the personal digital assistant is not less than the threshold, even for the same strength of the radio signal emitted from the wireless communication part, the strength of the radio signal received by the personal digital assistant is different according to the model of the personal digital assistant, the presence and material of a cover of the personal digital assistant, etc. Therefore, if the threshold is a fixed value, there is a risk that the user authentication cannot be performed, or a personal digital assistant outside the toilet room may be misauthenticated. In contrast, by making the threshold modifiable by the personal digital assistant, the radio wave strength (the threshold) at which the user authentication is performed can be set to an appropriate strength regardless of the model of the personal digital assistant, the presence and material of a cover of the personal digital assistant, etc. As a result, the user authentication not being performed can be suppressed, and misauthentication of a personal digital assistant outside the toilet room can be suppressed.

A ninth aspect is the toilet device of the seventh aspect, wherein the toilet device further includes a human body detection sensor configured to detect the user, and the controller performs the user authentication when the human body detection sensor detects the user and the distance between the personal digital assistant and the wireless communication part is not more than the prescribed distance.

According to the toilet device, the user authentication is performed when the human body detection sensor detects the user and the distance between the personal digital assistant and the wireless communication part is not more than the prescribed distance, so that misauthentication of a personal digital assistant outside the toilet room can be suppressed.

A tenth aspect is the toilet device of the ninth aspect, wherein the human body detection sensor includes a proximity sensor configured to detect an approach of the user, and a distance at which the proximity sensor can detect the user is different from the prescribed distance.

According to the toilet device, misauthentications can be further suppressed by setting the distance at which the proximity sensor can detect the user to be different from the prescribed distance.

An eleventh aspect is a method for controlling a toilet device; the toilet device is connectable with a personal digital assistant of a user by a wireless communication; the method includes a registration process and a user authentication process performed after the registration process; the registration process includes associating identification information identifying the personal digital assistant and user information identifying the user, and storing the identification information and the user information in a storage part; the user authentication process designates the user information and the identification information when a distance between the personal digital assistant and a wireless communication part of the toilet device becomes a prescribed distance or less; and the prescribed distance is modifiable by the personal digital assistant.

According to the method for controlling the toilet device, by making the prescribed distance modifiable by the personal digital assistant, the distance (the prescribed distance) within which the user authentication is performed can be set to an appropriate distance regardless of the physique of the user and the posture when the user utilizes the toilet device. The user authentication can be more reliably performed thereby.

A twelfth aspect is a recording medium; a control program is recorded in the recording medium; the control program is installed in a toilet device; the toilet device is connectable with a personal digital assistant of a user by a wireless communication; the control program is configured to cause the toilet device to perform a registration process, and a user authentication process performed after the registration process; the registration process includes associating identification information identifying the personal digital assistant and user information identifying the user, and storing the identification information and the user information in a storage part; the user authentication process designates the user information and the identification information when a distance between the personal digital assistant and a wireless communication part of the toilet device becomes a prescribed distance or less; and the prescribed distance is modifiable by the personal digital assistant.

According to the control program of the toilet device recorded in the recording medium, by making the prescribed distance modifiable by the personal digital assistant, the distance (the prescribed distance) within which the user authentication is performed can be set to an appropriate distance regardless of the physique of the user and the posture when the user utilizes the toilet device. The user authentication can be more reliably performed thereby.

Embodiments of the invention will now be described with reference to the drawings. Similar components in the drawings are marked with the same reference numerals; and a detailed description is omitted as appropriate.

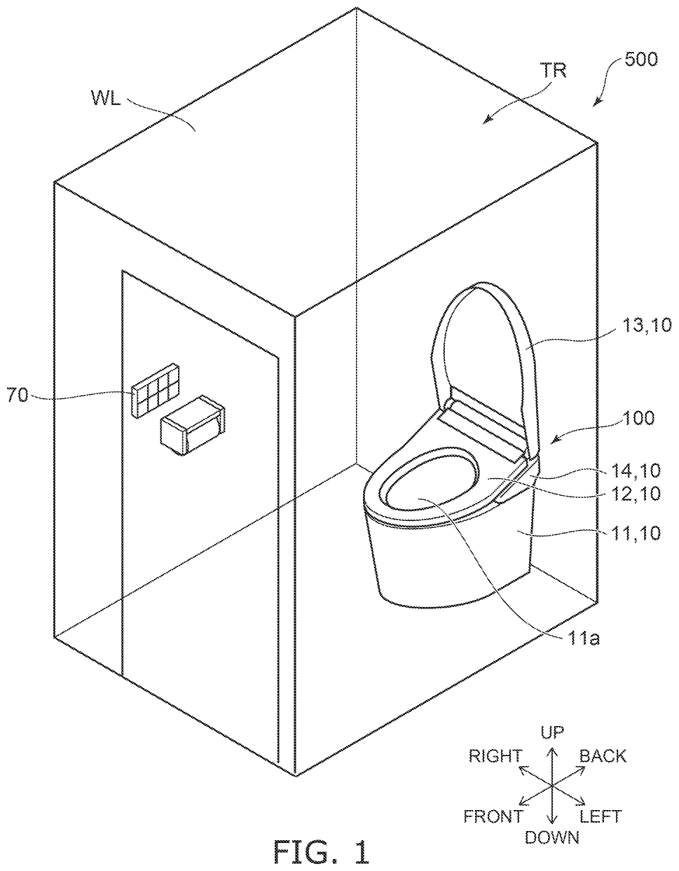

FIG. 1 is a perspective view illustrating a toilet system including a toilet device according to an embodiment.

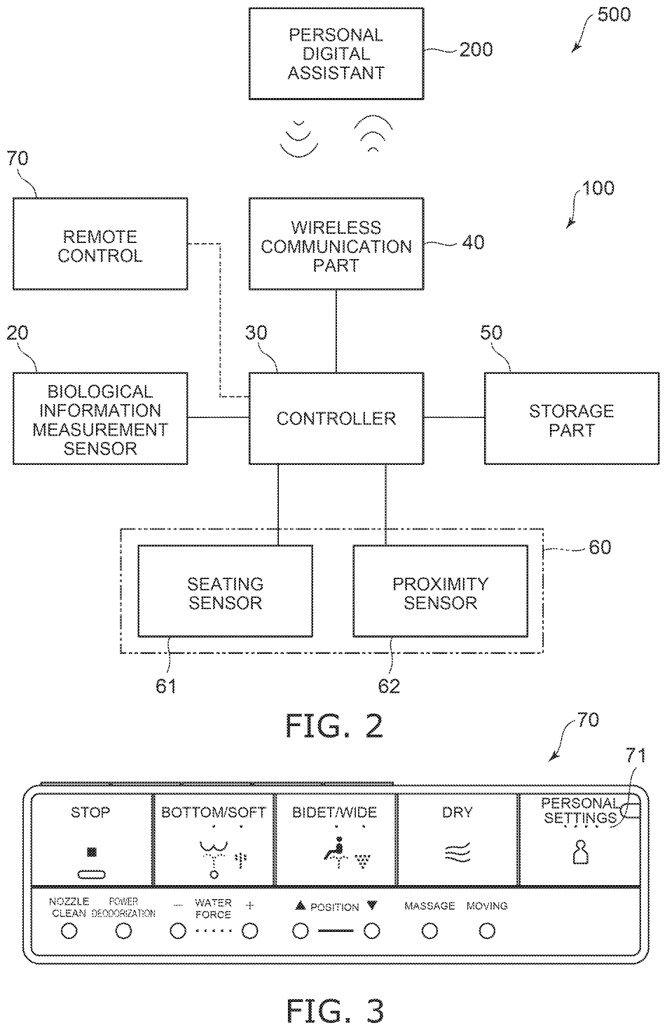

FIG. 2 is a block diagram illustrating the toilet system including the toilet device according to the embodiment.

FIG. 3 is a front view illustrating a remote control of the toilet device according to the embodiment.

As illustrated in FIGS. 1 to 3, the toilet system 500 according to the embodiment includes the toilet device 100 and a personal digital assistant 200.

The toilet device 100 includes a toilet unit 10, a biological information measurement sensor 20, a controller 30, a wireless communication part 40, a storage part 50, a human body detection sensor 60, and a remote control 70. The toilet device 100 is disposed inside a toilet room TR.

The toilet unit 10 includes a toilet 11, a toilet seat 12, a toilet lid 13, and a casing 14. The toilet 11 is a western-style sit-down toilet. The toilet 11 includes a concave bowl 11a that is recessed downward. The bowl 11a receives human waste, urine, etc., excreted from the user.

The toilet seat 12 and the toilet lid 13 each are pivotally supported to be openable and closable with respect to the casing 14. FIG. 1 illustrates a state in which the toilet seat 12 is closed and the toilet lid 13 is open. The toilet lid 13 covers the toilet seat 12 in the closed state from above. The toilet lid 13 is provided as necessary and is omissible.

In this specification, up, down, front, back, right, and left when viewed by a user seated on the toilet seat 12 with the user's back facing the toilet lid 13 are respectively taken as “up/above/higher than”, “down/downward/below/lower than”, “front/frontward”, “back/behind”, “right/rightward”, and “left/leftward”.

The casing 14 stores various functional units. For example, an automatic opening/closing unit automatically opening and closing the toilet seat 12 and/or the toilet lid 13, a private part washing unit including a nozzle for washing a private part of the user, a private part drying unit for drying a private part of the user, a deodorizing unit that deodorizes, etc., are stored inside the casing 14. These various functional units are provided as necessary and are omissible. The controller 30, the wireless communication part 40, the storage part 50, etc., also are stored inside the casing 14.

The biological information measurement sensor 20 measures biological information of the user. The biological information includes, for example, information related to the user's pulsatory motion, excrement state, etc. The biological information measurement sensor 20 is electrically connected with the controller 30. The biological information measurement sensor 20 outputs the measured biological information to the controller 30.

The biological information measurement sensor 20 includes, for example, at least one of a pulsatory motion sensor or an image sensor. For example, the pulsatory motion sensor is disposed in the toilet seat 12. The pulsatory motion sensor includes, for example, a laser sensor. For example, the pulsatory motion sensor measures the pulsatory motion of the user seated on the toilet seat 12 by using hemoglobin infrared reflection. For example, the image sensor is disposed inside the casing 14. The image sensor includes, for example, at least one of an area camera or a line sensor in which multiple pixels are arranged in a line shape.

The controller 30 is electrically connected with the biological information measurement sensor 20, the wireless communication part 40, the storage part 50, and the human body detection sensor 60. The controller 30 acquires the measurement result (the biological information) of the biological information measurement sensor 20 and the detection result of the human body detection sensor 60. The controller 30 controls the biological information measurement sensor 20, the wireless communication part 40, and the storage part 50. For example, the controller 30 controls the start and stop of the measurement of the biological information by the biological information measurement sensor 20. The controller 30 is, for example, a control circuit including an IC element. One or multiple controllers 30 may be included. When multiple controllers 30 are included, the controllers 30 may be concentrated or dispersed. The controller 30 includes, for example, a main body controller (a DC controller), a sensor controller (a processor unit), and a wireless controller. For example, the start and stop of the measurement of the biological information measurement sensor 20 are controlled by the main body controller and/or the sensor controller.

The controller 30 acquires analysis information by analyzing the biological information acquired from the biological information measurement sensor 20. For example, by analyzing the information (the biological information) related to the pulsatory motion of the user acquired from the pulsatory motion sensor, the controller 30 can acquire (estimate) information related to the vascular age, lower limb blood circulation status, fitness level, basal metabolism, body water level, body clock, normal heart rate, etc. For example, the controller 30 also can acquire (estimate) information related to the state of the intestinal environment by using image data acquired from the image sensor to analyze the state (the shape, color, amount, etc.) of the excrement and by further analyzing the information (the biological information) related to the state of the excrement. The analysis of the biological information is performed as necessary and is partially or entirely omissible. For example, the analysis of the biological information is performed by the main body controller and/or the sensor controller.

The controller 30 transmits the analysis information to the personal digital assistant 200 of the user via the wireless communication part 40. When analysis of the biological information is omitted, the controller 30 transmits, for example, the biological information to the personal digital assistant 200 of the user via the wireless communication part 40. The controller 30 also stores the analysis information or biological information in the storage part 50. The controller 30 also erases the analysis information or biological information stored in the storage part 50 from the storage part 50. The analysis of the biological information may be performed by the personal digital assistant 200 or may be performed by a cloud server via the personal digital assistant 200. The controller 30 also may transmit the analysis information or biological information to the personal digital assistant 200 via a cloud server.

The wireless communication part 40 is configured to connect the controller 30 and the personal digital assistant 200 by wireless communication. The wireless communication part 40 is electrically connected with the controller 30. The wireless communication part 40 is, for example, a communication module configured to communicate using a Bluetooth (registered trademark) standard such as BLE (Bluetooth Low Energy), etc. For example, the wireless communication part 40 is configured to connect the controller 30 and the personal digital assistant 200 positioned inside the toilet room TR by wireless communication. For example, the connection between the controller 30 and the personal digital assistant 200 is disconnected when the personal digital assistant 200 is outside the toilet room TR. Also, the connection between the controller 30 and the personal digital assistant 200 is disconnected when, for example, a disconnect request signal is transmitted from the personal digital assistant 200. The connection between the toilet device 100 (the controller 30) and the personal digital assistant 200 is described below.

The storage part 50 is configured to store the information output from the controller 30. The storage part 50 is electrically connected with the controller 30. One or multiple storage parts 50 may be included. When multiple storage parts 50 are included, the storage parts 50 may be concentrated or dispersed. The storage part 50 includes, for example, a main body storage part, a sensor storage part, and a wireless storage part. The main body storage part is electrically connected with the main body controller. The sensor storage part is electrically connected with the sensor controller. The wireless storage part is electrically connected with the wireless controller. For example, the storage part 50 stores the analysis information or the biological information. For example, in the state in which the user authentication is performed, the storage part 50 associates and stores the analysis information or biological information and user information identifying the user. More specifically, for example, information that is associated with the analysis information and the user information is stored in the sensor storage part. The user authentication is described below.

The storage part 50 associates and stores identification information identifying the personal digital assistant 200 and the user information identifying the user. More specifically, for example, information in which the identification information and the user information are associated is stored in the wireless storage part. A registration mode of associating and storing the identification information and the user information is described below. The identification information includes, for example, at least one of an ID number, a BD address, a MAC address, or pairing information (cryptographic information shared by the toilet device 100 and the control program of the personal digital assistant 200 when pairing). It is favorable for the identification information to include the pairing information. For example, the personal digital assistant 200 that receives an advertisement signal from the toilet device 100 transmits a wireless communication request to the toilet device 100; the toilet device 100 that receives the wireless communication request permits wireless communication; and communication is established. When communicating with the personal digital assistant 200 of a user 1, if the pairing information and the user number are transmitted with the wireless communication request, the toilet device 100 can determine, based on the user No. 1 being paired, that the connection request is from the personal digital assistant 200 of the user 1.

The human body detection sensor 60 detects the state of the user. The human body detection sensor 60 includes, for example, at least one of a seating sensor 61 or a proximity sensor 62. For example, the seating sensor 61 is disposed at the toilet seat 12. The seating sensor 61 includes, for example, a capacitive sensor. For example, the seating sensor 61 detects the seating of the user on the toilet seat 12 and the user leaving the toilet seat 12. For example, the proximity sensor 62 is disposed inside the casing 14. The proximity sensor 62 includes, for example, at least one of a radio wave sensor or an infrared sensor. For example, the proximity sensor 62 detects the user approaching the toilet unit 10 and the user moving away from the toilet unit 10. For example, the proximity sensor 62 detects the user entering the toilet room TR and the user exiting the toilet room TR. The proximity sensor 62 may be disposed outside the casing 14.

For example, the remote control 70 is disposed at a wall surface WL of the toilet room TR. The remote control 70 is an operation unit for remotely operating various functional units stored inside the casing 14. For example, the remote control 70 transmits, to the controller 30, signals for operating the various functional units based on operation input of the remote control 70 by the user. When receiving the signals from the remote control 70, the controller 30 controls the various functional units based on the signals. For example, the remote control 70 is connected with the controller 30 by wireless communication. For example, the wireless communication part that connects the remote control 70 and the controller 30 is provided separately from the wireless communication part 40.

The remote control 70 includes, for example, an authentication switch 71 for transmitting an authentication signal. When an operation input of the authentication switch 71 is performed, the remote control 70 transmits the authentication signal to the controller 30. For example, the controller 30 can perform user authentication based on the authentication signal from the remote control 70. The user authentication is described below.

The personal digital assistant 200 is, for example, a smartphone, a tablet terminal, or the like possessed by the user. However, the personal digital assistant 200 is not limited to such terminals; any terminal that can be carried by the user may be used.

A control program (application software) for connecting with the toilet device 100 and displaying information (e.g., analysis information) transmitted from the toilet device 100 is preinstalled in the personal digital assistant 200.

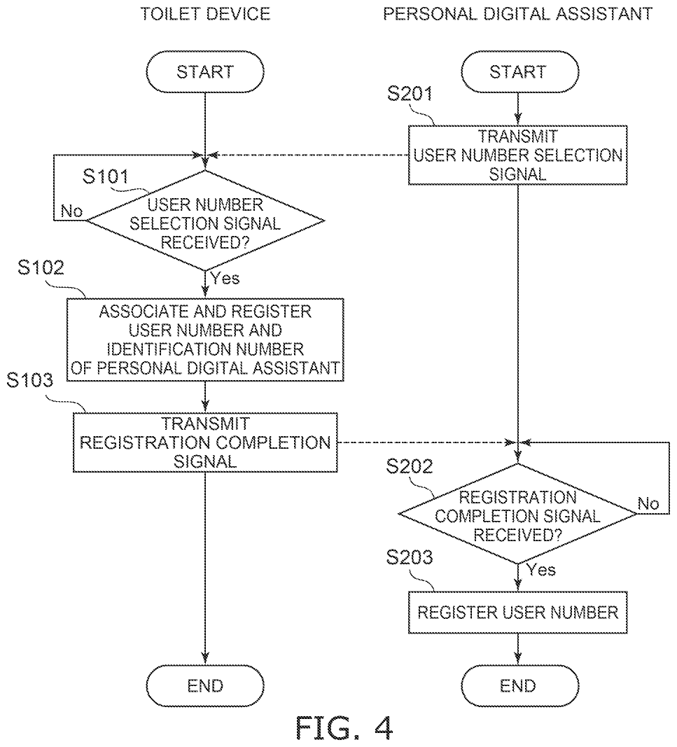

FIG. 4 is a flowchart illustrating an example of an operation of a registration mode of the toilet device and the personal digital assistant according to the embodiment.

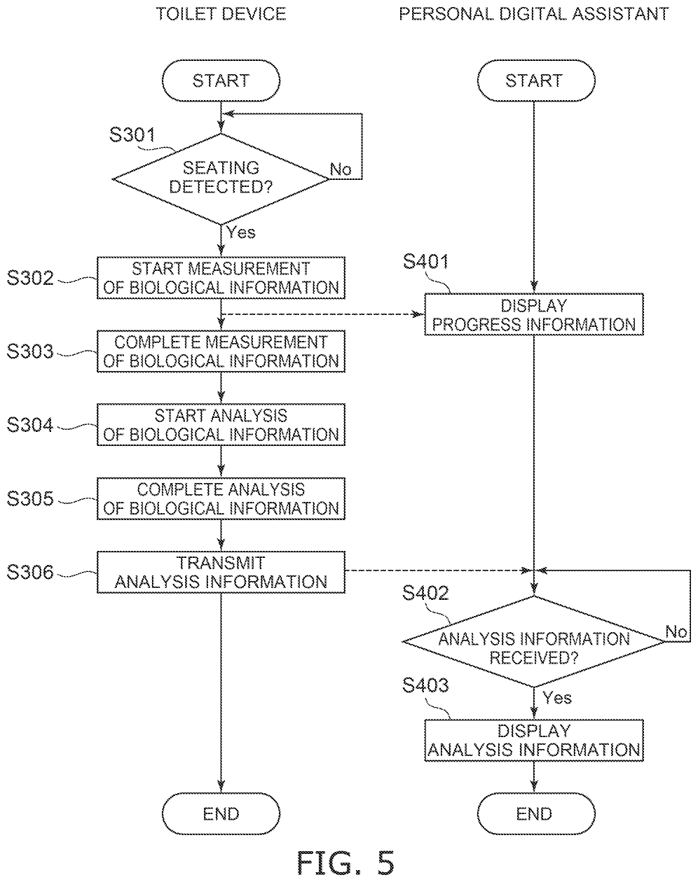

FIG. 5 is a flowchart illustrating an example of an operation of a use mode of the toilet device and the personal digital assistant according to the embodiment.

As illustrated in FIGS. 4 and 5, the toilet device 100 (the controller 30) includes the registration mode and the use mode.

The registration mode is a mode in which user registration can be performed. User registration is the operation of associating the identification information identifying the personal digital assistant 200 and the user information (e.g., the user number) identifying the user, and storing the identification information and the user information in the storage part 50. The registration mode is performed in a state in which the toilet device 100 (the controller 30) and the personal digital assistant 200 are connected.

The use mode is a mode in which measurement of the biological information, analysis of the biological information, and transmission of the analysis information or biological information can be performed. User authentication can be performed in the use mode when user registration has already been performed. The user authentication is the designation of the user information (the user number) of the user using the toilet device 100 and the identification information of the personal digital assistant 200 of the user. By performing the user authentication, for example, the analysis information or biological information can be associated with the user information (the user number) and the identification information of the personal digital assistant 200 of the user and stored in the storage part 50. Also, by performing the user authentication, for example, the toilet device 100 and the personal digital assistant 200 designated by the user authentication can be selectively connected.

For example, the user authentication is performed by connecting the toilet device 100 (the controller 30) and the personal digital assistant 200. In such a case, the personal digital assistant 200 that is connected with the toilet device 100 (the controller 30) transmits, to the toilet device 100, an authentication signal including the user information (the user number) registered in the personal digital assistant 200. When receiving the authentication signal, the toilet device 100 determines that the user of the user information (the user number) included in the authentication signal is using the toilet device 100. As a result, the user information (the user number) of the user using the toilet device 100 and the identification information of the personal digital assistant 200 of the user can be designated. The user authentication using the personal digital assistant 200 is described below.

For example, the user authentication may be performed by an operation input of the authentication switch 71 of the remote control 70. In such a case, when the operation input of the authentication switch 71 (e.g., the selection of the user number) is performed, the remote control 70 transmits an authentication signal including the user information (the user number) to the toilet device 100. When receiving the authentication signal, the toilet device 100 determines that the user of the user information (the user number) included in the authentication signal is using the toilet device 100. As a result, the user information (the user number) of the user using the toilet device 100 and the identification information of the personal digital assistant 200 of the user can be designated. The user authentication using the remote control 70 is described below.

The registration mode will now be described in more detail.

In the registration mode as illustrated in FIG. 4, the toilet device 100 (the controller 30) determines whether or not a user number selection signal is received (step S101). The toilet device 100 (the controller 30) repeats step S101 until the user number selection signal is received (step S101: No). The user number selection signal indicates a user number selected by the personal digital assistant 200.

When the user number selection signal is received (step S101: Yes), the toilet device 100 (the controller 30) associates the selected user number and the identification number of the personal digital assistant 200 transmitting the user number selection signal and stores (registers) the selected user number and the identification number in the storage part 50 (step S102).

The user number and the identification number are associated and registered (step S102); and the toilet device 100 (the controller 30) transmits a registration completion signal to the personal digital assistant 200 (step S103). The registration completion signal indicates that the registration is completed.

When the user number is selected by the personal digital assistant 200, the personal digital assistant 200 transmits the user number selection signal to the toilet device 100 (step S201).

When the user number selection signal is transmitted to the toilet device 100 (step S201), the personal digital assistant 200 determines whether or not the registration completion signal is received from the toilet device 100 (step S202). The personal digital assistant 200 repeats step S202 until the registration completion signal is received (step S202: No).

When the registration completion signal is received (step S202: Yes), the personal digital assistant 200 stores (registers) the registered user number (step S203).

The use mode will now be described in more detail.

In the use mode as illustrated in FIG. 5, the toilet device 100 (the controller 30) determines whether or not the seating sensor 61 detects the seating of the user on the toilet seat 12 (step S301). The toilet device 100 (the controller 30) repeats step S301 until the seating sensor 61 detects the seating of the user on the toilet seat 12 (step S301: No).

When the seating sensor 61 detects the seating of the user on the toilet seat 12 (step S301: Yes), the toilet device 100 (the controller 30) starts the measurement of biological information (step S302). As described above, the measurement of the biological information is performed by the biological information measurement sensor 20 such as a pulsatory motion sensor, an image sensor, etc.

When the measurement of the biological information is completed (step S303), the toilet device 100 (the controller 30) starts the analysis of the biological information (step S304). As described above, the analysis of the biological information is performed by the controller 30. When the user is detected leaving the toilet seat 12 before the measurement is completed, for example, the toilet device 100 (the controller 30) ends the measurement of the biological information partway, and starts the analysis of the biological information.

When the analysis of the biological information is completed (step S305), the toilet device 100 (the controller 30) transmits the analysis information obtained by the analysis to the personal digital assistant 200 (step S306). As described above, the transmission of the analysis information is performed by the controller 30 via the wireless communication part 40.

For example, the personal digital assistant 200 displays progress information of the measurement of the biological information (step S401). The display of the progress information is described below.

The personal digital assistant 200 determines whether or not the analysis information is received (step S402). The personal digital assistant 200 repeats step S402 until the analysis information is received (step S402: No).

When the analysis information is received (step S402: Yes), the personal digital assistant 200 displays the analysis information (step S403). The personal digital assistant 200 may store the analysis information to be able to display the analysis information even after the connection between the toilet device 100 (the controller 30) and the personal digital assistant 200 is disconnected.

In step S301, the toilet device 100 (the controller 30) may determine whether or not the proximity sensor 62 has detected the approach of the user to the toilet unit 10, or may determine whether or not the proximity sensor 62 has detected the user entering the toilet room TR. That is, the trigger of the measurement start of the biological information of the toilet device 100 (the controller 30) may be the seating of the user on the toilet seat 12, the approach of the user to the toilet unit 10, or the user entering the toilet room TR. The toilet device 100 (the controller 30) may start the measurement of the biological information when the user authentication is completed. The toilet device 100 (the controller 30) may operate a line sensor in a power saving mode and then start the measurement of the biological information when a falling object is detected. Or, the toilet device 100 (the controller 30) may operate a laser sensor in a power saving mode and then start the measurement of the biological information when an output change is detected.

The connection between the toilet device 100 and the personal digital assistant 200 will now be described in more detail.

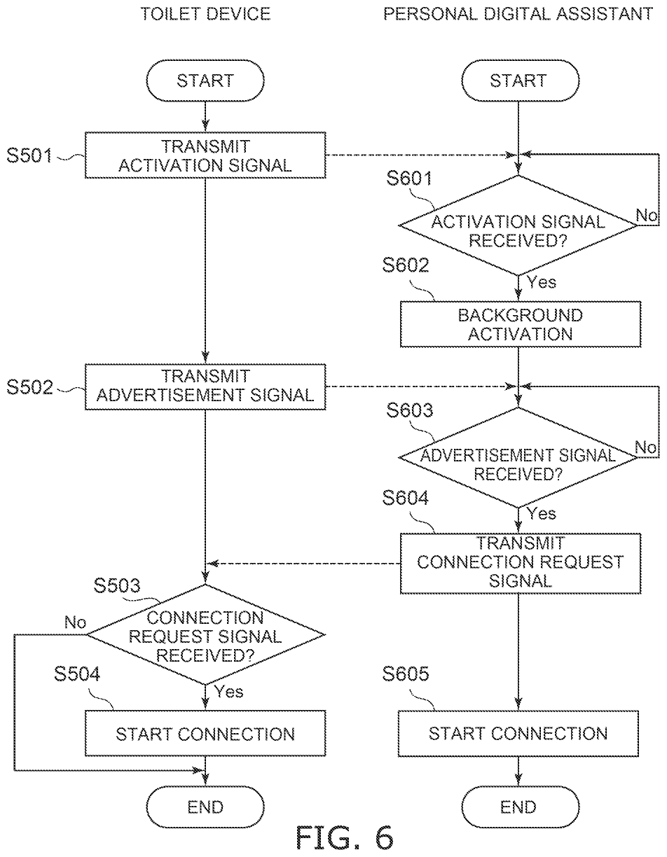

FIG. 6 is a flowchart illustrating an example of an operation when connecting the toilet device and the personal digital assistant according to the embodiment.

As illustrated in FIG. 6, the toilet device 100 (the controller 30) transmits an activation signal to the personal digital assistant 200 (step S501). The activation signal is a signal to set the personal digital assistant 200 to a background active state. The background active state is a state in which an advertisement signal can be received.

When transmitting the activation signal (step S501), the toilet device 100 (the controller 30) transmits an advertisement signal to the personal digital assistant 200 (step S502). The advertisement signal is a signal for causing the personal digital assistant 200 to start a connection with the toilet device 100 (the controller 30).

When transmitting the advertisement signal (step S502), the toilet device 100 (the controller 30) determines whether or not a connection request signal is received (step S503). The connection request signal is a signal for causing the toilet device 100 (the controller 30) to start a connection with the personal digital assistant 200.

When the connection request signal is received (step S503: Yes), the toilet device 100 (the controller 30) starts the connection with the personal digital assistant 200 (step S504). When the connection request signal is not received (step S503: No), the toilet device 100 (the controller 30) does not perform step S504. That is, when the connection request signal is not received (step S503: No), the toilet device 100 (the controller 30) does not start a connection with the personal digital assistant 200.

The personal digital assistant 200 determines whether or not the activation signal is received (step S601). The personal digital assistant 200 repeats step S601 until the activation signal is received (step S601: No).

When the activation signal is received (step S601: Yes), the personal digital assistant 200 is set to a background active state (step S602).

When set to the background active state (step S602), the personal digital assistant 200 determines whether or not the advertisement signal is received (step S603). The personal digital assistant 200 repeats step S603 until the advertisement signal is received (step S603: No).

When the advertisement signal is received (step S603: Yes), the personal digital assistant 200 transmits a connection request signal to the toilet device 100 (the controller 30) (step S604) and starts the connection with the toilet device 100 (the controller 30) (step S605).

The user authentication using the personal digital assistant 200 will now be described in more detail.

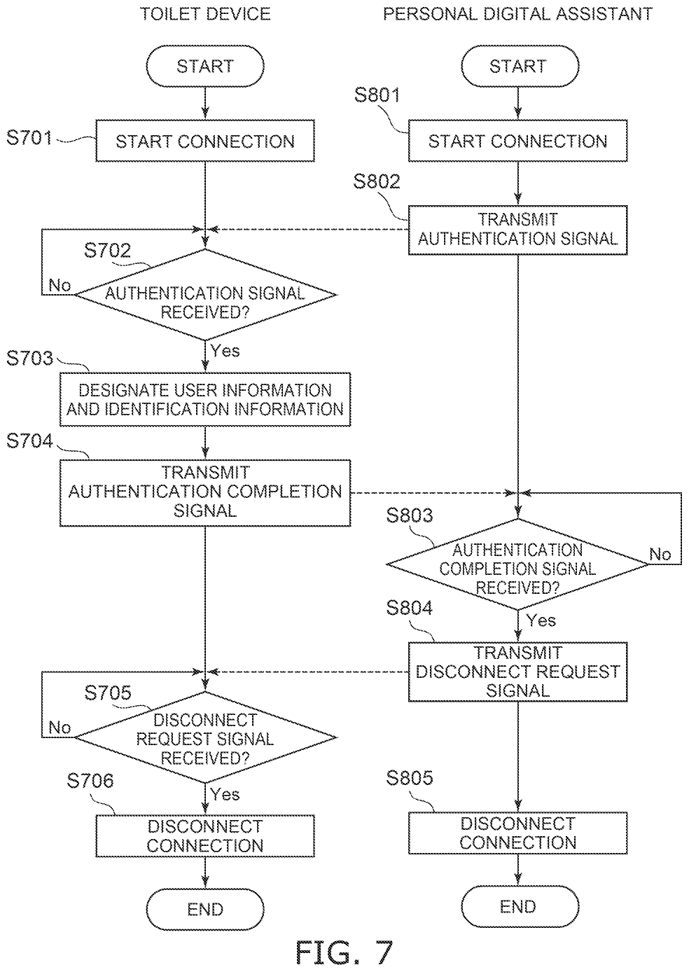

FIG. 7 is a flowchart illustrating an example of an operation in the user authentication of the toilet device and the personal digital assistant according to the embodiment.

As illustrated in FIG. 7, when starting the connection with the personal digital assistant 200 (step S701), the toilet device 100 (the controller 30) determines whether or not an authentication signal is received (step S702). The toilet device 100 (the controller 30) repeats step S702 until the authentication signal is received (step S702: No). The authentication signal is, for example, a signal that includes the user information (the user number).

When the authentication signal is received (step S702: Yes), the toilet device 100 (the controller 30) designates the user information and the identification information based on the authentication signal (step S703). More specifically, the toilet device 100 (the controller 30) designates the user information included in the authentication signal and designates the personal digital assistant 200 having the identification information associated with the user information included in the authentication signal.

When the user information and the identification information are designated (step S703), the toilet device 100 (the controller 30) transmits an authentication completion signal to the personal digital assistant 200 (step S704). The authentication completion signal indicates that the user authentication is completed.

When transmitting the authentication completion signal (step S704), the toilet device 100 (the controller 30) determines whether or not a disconnect request signal is received (step S705). The toilet device 100 (the controller 30) repeats step S705 until the disconnection request signal is received (step S705: No). The disconnection request signal is a signal for causing the toilet device 100 (the controller 30) to disconnect the connection with the personal digital assistant 200.

When the disconnection request signal is received (step S705: Yes), the toilet device 100 (the controller 30) disconnects the connection with the personal digital assistant 200 (step S706).

When starting the connection with the toilet device 100 (the controller 30) (step S801), the personal digital assistant 200 transmits the authentication signal (step S802).

When transmitting the authentication signal (step S802), the personal digital assistant 200 determines whether or not the authentication completion signal is received (step S803). The personal digital assistant 200 repeats step S803 until the authentication completion signal is received (step S803: No).

When the authentication completion signal is received (step S803: Yes), the personal digital assistant 200 transmits the disconnection request signal (step S804) and disconnects the connection with the toilet device 100 (the controller 30) (step S805).

Thus, after performing the registration mode (i.e., in the state in which the identification information and the user information are associated and stored in the storage part 50), the toilet device 100 (the controller 30) can perform user authentication in the use mode to designate the user information and the identification information when receiving the authentication signal.

The user authentication using the remote control 70 will now be described in more detail.

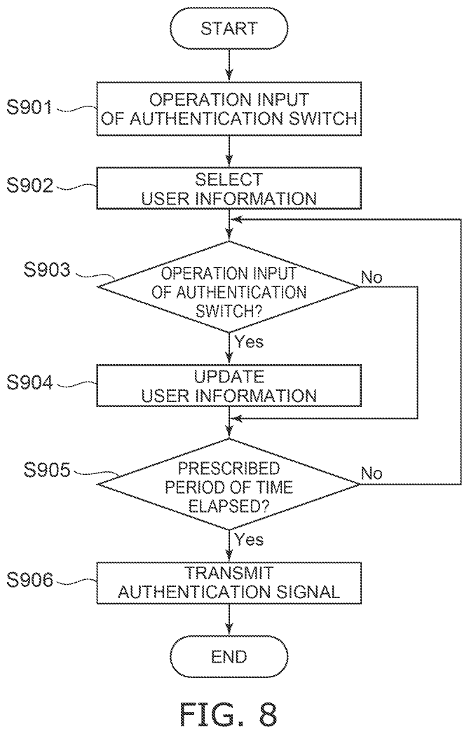

FIG. 8 is a flowchart illustrating an example of an operation in the user authentication by the remote control of the toilet device according to the embodiment.

As illustrated in FIG. 8, when an operation input of the authentication switch 71 is performed (step S901), the remote control 70 selects user information (step S902). More specifically, the remote control 70 selects, for example, the user number 1 for the first operation input.

When selecting the user information (step S902), the remote control 70 determines whether or not a further operation input of the authentication switch 71 is performed (step S903). When a further operation input of the authentication switch 71 has been performed (step S903: Yes), the remote control 70 updates the user information (step S904). More specifically, for example, the remote control 70 selects the user number 2 when a second operation input is performed. For example, the remote control 70 selects the user number 3 when a third operation input is performed. For example, the remote control 70 selects the user number N when the Nth operation input (N being a natural number) is performed. In other words, for example, the remote control 70 adds 1 to the numerical value of the user number when the operation input is performed. For example, the remote control 70 selects the user number 1 when a further operation input is performed in a state in which the numerical value of the user number has reached an upper limit.

When updating the user information (step S904), the remote control 70 determines whether or not a prescribed period of time has elapsed from the previous operation input (step S905). When the prescribed period of time has not elapsed from the previous operation input (step S905: No), the remote control 70 returns to step S903.

When a further operation input is not performed (step S903: No), the remote control 70 performs step S905 without performing step S904. The remote control 70 repeats step S903, step S904, and step S905 until the prescribed period of time has elapsed from the previous operation input.

When the prescribed period of time has elapsed from the previous operation input (step S905: Yes), the remote control 70 finalizes the user information included in the authentication signal and transmits the authentication signal to the controller 30 (step S906). More specifically, the remote control 70 transmits the authentication signal including the finally-selected user number.

Thus, by including the remote control 70 that transmits the authentication signal, compared to when, for example, the authentication signal is transmitted by operating an operation unit or the like disposed in a location (e.g., the toilet unit 10, etc.) other than the remote control 70, the transmission of the authentication signal (the user authentication) can be used in a form close to normal use which is easier to use.

The user information for the user authentication can be switched by the number of times one authentication switch 71 is operated by selecting the user information to be designated by the authentication signal based on the number of times that the operation input of the authentication switch 71 is performed until the prescribed period of time has elapsed, and then transmitting the authentication signal when the prescribed period of time has elapsed. Misauthentications can be suppressed thereby.

The progress check of the measurement of the biological information will now be described in more detail.

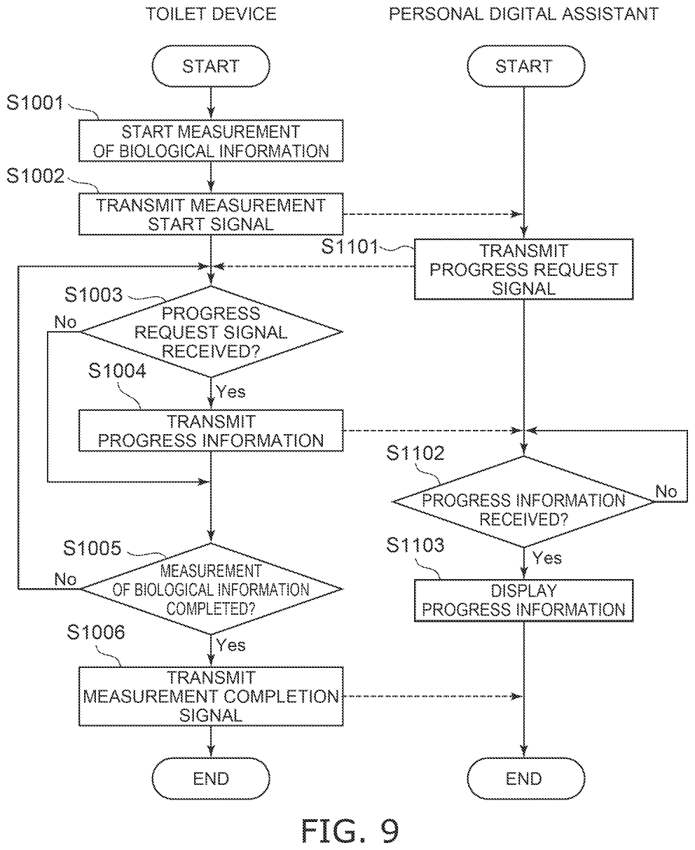

FIG. 9 is a flowchart illustrating an example of an operation of the use mode of the toilet device according to the embodiment.

As illustrated in FIG. 9, when starting the measurement of the biological information (step S1001), the toilet device 100 (the controller 30) transmits a measurement start signal to the personal digital assistant 200 (step S1002). The measurement start signal indicates that the measurement of the biological information has started.

When transmitting the measurement start signal (step S1002), the toilet device 100 (the controller 30) determines whether or not a progress request signal is received (step S1003). The progress request signal is a signal for causing the toilet device 100 (the controller 30) to transmit progress information. The progress information indicates the progress of the measurement of the biological information. When receiving the progress request signal (step S1003: Yes), the toilet device 100 (the controller 30) transmits the progress information (step S1004).

When transmitting the progress information (step S1004), the toilet device 100 (the controller 30) determines whether or not the measurement of the biological information is completed (step S1005). When the progress request signal is not received (step S1003: No), the toilet device 100 (the controller 30) performs step S1005 without performing step S1004.

When the measurement of the biological information is not completed (step S1005: No), the toilet device 100 (the controller 30) returns to step S1003. The toilet device 100 (the controller 30) repeats step S1003, step S1004, and step S1005 until the measurement of the biological information is completed.

When the measurement of the biological information is completed (step S1005: Yes), the toilet device 100 (the controller 30) transmits a measurement completion signal to the personal digital assistant 200 (step S1006). The measurement completion signal indicates that the measurement of the biological information is completed.

When receiving the measurement start signal, the personal digital assistant 200 is enabled to transmit the progress request signal until the measurement completion signal is received. For example, the personal digital assistant 200 transmits the progress request signal to the toilet device 100 (the controller 30) (step S1101) when an operation input to display the progress information is input to the personal digital assistant 200 while the personal digital assistant 200 is enabled to transmit the progress request signal.

When transmitting the progress request signal (step S1101), the personal digital assistant 200 determines whether or not the progress information is received (step S1102). The personal digital assistant 200 repeats step S1102 until the progress information is received (step S1102: No).

When the progress information is received (step S1102: Yes), the personal digital assistant 200 displays the progress information (step S1103).

The personal digital assistant 200 maintains the state of being enabled to transmit the progress request signal until the measurement completion signal is received. When the measurement completion signal is received, the personal digital assistant 200 is set to the state of being enabled to transmit the progress request signal.

In the example, when the measurement start signal is received, the progress information is displayed by the personal digital assistant 200 only while the measurement is being performed by setting the personal digital assistant 200 to a state of being enabled to transmit the progress request signal until the measurement completion signal is received; however, the progress information may be displayed by the personal digital assistant 200 only while the measurement is being performed by enabling the toilet device 100 (the controller 30) to transmit the progress information only while the measurement is being performed. That is, when the measurement is not being performed, the toilet device 100 (the controller 30) may not transmit the progress information even when the progress request signal is received. In such a case, step S1102 and step S1006 are omissible.

The user authentication using the personal digital assistant 200 will now be described in more detail.

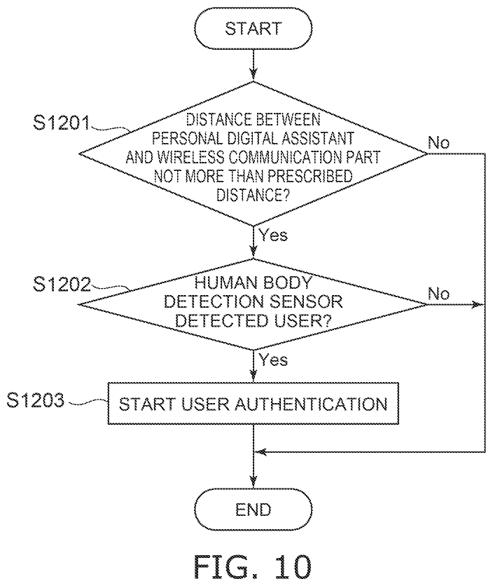

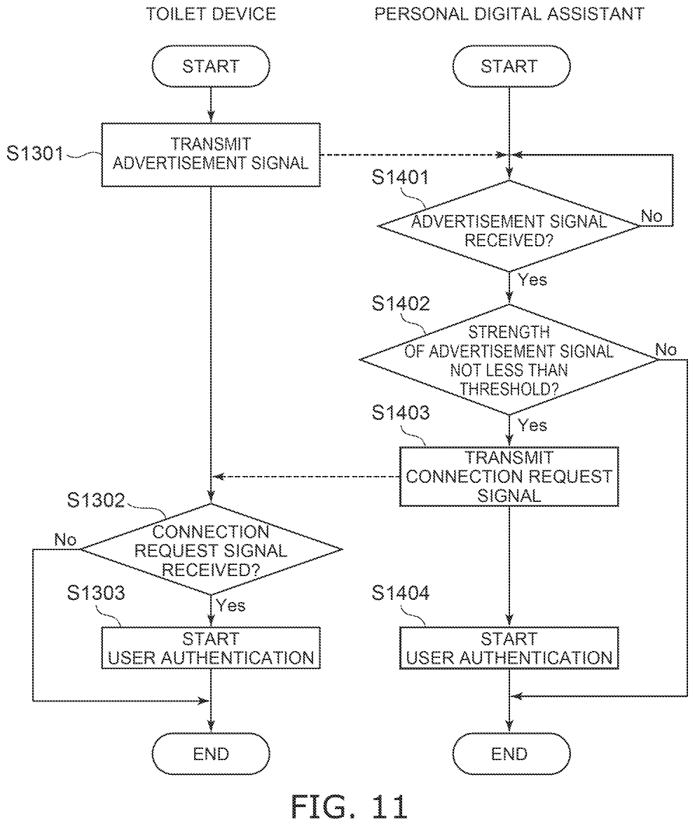

FIG. 10 is a flowchart illustrating an example of an operation in the user authentication of the toilet device according to the embodiment.

As illustrated in FIG. 10, the toilet device 100 (the controller 30) determines whether or not the distance between the personal digital assistant 200 and the wireless communication part 40 is not more than a prescribed distance (step S1201).

When the distance between the personal digital assistant 200 and the wireless communication part 40 is not more than the prescribed distance (step S1201: Yes), the toilet device 100 (the controller 30) determines whether or not the human body detection sensor 60 detects the user (step S1202). For example, in step S1202, the toilet device 100 (the controller 30) determines that the human body detection sensor 60 detects the user when the seating sensor 61 detects the seating of the user on the toilet seat 12. For example, in step S1202, the toilet device 100 (the controller 30) determines that the human body detection sensor 60 detects the user when the proximity sensor 62 detects the approach of the user to the toilet unit 10. For example, in step S1202, the toilet device 100 (the controller 30) determines that the human body detection sensor 60 detects the user when the proximity sensor 62 detects the user entering the toilet room TR.

When the human body detection sensor 60 detects the user (step S1202: Yes), the toilet device 100 (the controller 30) starts the user authentication (step S1203).

When the distance between the personal digital assistant 200 and the wireless communication part 40 is greater than the prescribed distance (step S1201: No), the toilet device 100 (the controller 30) does not perform step S1202 and step S1203. That is, when the distance between the personal digital assistant 200 and the wireless communication part 40 is greater than the prescribed distance (step S1201: No), the toilet device 100 (the controller 30) does not start the user authentication.

When the human body detection sensor 60 does not detect the user (step S1202: No), the toilet device 100 (the controller 30) does not perform step S1203. That is, when the human body detection sensor 60 does not detect the user (step S1202: No), the toilet device 100 (the controller 30) does not start the user authentication.

The determination of whether or not the distance between the personal digital assistant 200 and the wireless communication part 40 is not more than the prescribed distance may be performed by the toilet device 100 (the controller 30) or may be performed by the personal digital assistant 200.

The prescribed distance is modifiable by the personal digital assistant 200. For example, the user can modify the prescribed distance stored in the toilet device 100 or the personal digital assistant 200 to any value by an operation input of the personal digital assistant 200.

Thus, by making the prescribed distance modifiable by the personal digital assistant 200, the distance (the prescribed distance) within which the user authentication is performed can be set to an appropriate distance regardless of the physique of the user and the posture when the user utilizes the toilet device 100. The user authentication can be more reliably performed thereby.

For example, whether or not the distance between the personal digital assistant 200 and the wireless communication part 40 is not more than the prescribed distance is determined by whether or not the strength of a radio signal (e.g., the advertisement signal) emitted from the wireless communication part 40 and received by the personal digital assistant 200 is not less than a threshold. For example, when the strength of the radio signal emitted from the wireless communication part 40 and received by the personal digital assistant 200 is less than the threshold, the distance between the personal digital assistant 200 and the wireless communication part 40 is determined to be greater than the prescribed distance. For example, when the strength of the radio signal emitted from the wireless communication part 40 and received by the personal digital assistant 200 is not less than the threshold, the distance between the personal digital assistant 200 and the wireless communication part 40 is determined to be not more than the prescribed distance. The determination of whether or not the strength of the radio signal emitted from the wireless communication part 40 and received by the personal digital assistant 200 is not less than the threshold may be performed by the toilet device 100 (the controller 30) or may be performed by the personal digital assistant 200.

Thus, for example, the toilet device 100 (the controller 30) does not start the user authentication when the strength of the radio signal emitted from the wireless communication part 40 and received by the personal digital assistant 200 is less than the threshold; and the toilet device 100 (the controller 30) starts the user authentication when the strength of the radio signal emitted from the wireless communication part 40 and received by the personal digital assistant 200 is not less than the threshold.

Whether or not the distance between the personal digital assistant 200 and the wireless communication part 40 is not more than the prescribed distance is determined by whether or not the strength of the radio signal emitted from the wireless communication part 40 and received by the personal digital assistant 200 is not less than the threshold, making it easy to determine whether or not the distance between the personal digital assistant 200 and the wireless communication part 40 is not more than the prescribed distance by using the strength of the radio signal received by the personal digital assistant 200.

The threshold is modifiable by the personal digital assistant 200. For example, the user can modify the threshold stored in the toilet device 100 or the personal digital assistant 200 to any value by an operation input of the personal digital assistant 200.

When determining whether or not the strength of the radio signal emitted from the wireless communication part 40 and received by the personal digital assistant 200 is not less than the threshold, even for the same strength of the radio signal emitted from the wireless communication part 40, the strength of the radio signal received by the personal digital assistant 200 is different according to the model of the personal digital assistant 200, the presence and material of a cover of the personal digital assistant 200, etc. Therefore, if the threshold is set to a fixed value, there is a risk that the user authentication cannot be performed, or a personal digital assistant 200 that is outside the toilet room TR may be misauthenticated. In contrast, by making the threshold modifiable by the personal digital assistant 200, the radio wave strength (the threshold) at which the user authentication is performed can be set to an appropriate strength regardless of the model of the personal digital assistant 200, the presence and material of a cover of the personal digital assistant 200, etc. As a result, the user authentication not being performed can be suppressed, and misauthentication of a personal digital assistant 200 outside the toilet room TR can be suppressed.

By performing the user authentication when the human body detection sensor 60 detects the user and the distance between the personal digital assistant 200 and the wireless communication part 40 is not more than the prescribed distance, misauthentication of a personal digital assistant 200 outside the toilet room TR can be suppressed.

Whether or not the distance between the personal digital assistant 200 and the wireless communication part 40 is not more than the prescribed distance may be determined by whether or not the distance between the personal digital assistant 200 and the wireless communication part 40 measured by, for example, GPS (Global Positioning System), etc., is not more than the prescribed distance.

Step S1202 is performed as necessary and is omissible. When step S1202 is omitted, the toilet device 100 (the controller 30) starts the user authentication (step S1203) when the distance between the personal digital assistant 200 and the wireless communication part 40 is not more than the prescribed distance (step S1201: Yes).

A case will now be described where the personal digital assistant 200 performs the determination of whether or not the strength of the radio signal emitted from the wireless communication part 40 and received by the personal digital assistant 200 is not less than the threshold.

FIG. 11 is a flowchart illustrating an example of an operation in the user authentication of the toilet device and the personal digital assistant according to the embodiment.

As illustrated in FIG. 11, when transmitting the advertisement signal (step S1301), the toilet device 100 (the controller 30) determines whether or not a connection request signal is received (step S1302).

When receiving the connection request signal (step S1302: Yes), the toilet device 100 (the controller 30) starts a connection with the personal digital assistant 200 (step S1303). When a connection request signal is not received (step S1302: No), the toilet device 100 (the controller 30) does not perform step S1303. That is, when the connection request signal is not received (step S1302: No), the toilet device 100 (the controller 30) does not start the user authentication.

The personal digital assistant 200 determines whether or not the advertisement signal is received (step S1401). The personal digital assistant 200 repeats step S1401 until the advertisement signal is received (step S1401: No).

When receiving the advertisement signal (step S1401: Yes), the personal digital assistant 200 determines whether or not the strength of the received advertisement signal is not less than a threshold (step S1402).

When the strength of the received advertisement signal is not less than the threshold (step S1402: Yes), the personal digital assistant 200 transmits the connection request signal to the toilet device 100 (the controller 30) (step S1403) and starts the user authentication (step S1404). Step S1404 is performed as necessary and is omissible.

When the strength of the received advertisement signal is less than the threshold (step S1402: No), the personal digital assistant 200 does not perform step S1403 and step S1404. That is, when the strength of the received advertisement signal is less than the threshold (step S1402: No), the personal digital assistant 200 does not transmit the connection request signal to the toilet device 100 (the controller 30) and does not start the user authentication.

Thus, the determination of whether or not the strength of the radio signal emitted from the wireless communication part 40 and received by the personal digital assistant 200 is not less than the threshold is performed by, for example, the personal digital assistant 200. That is, the determination of whether or not the distance between the personal digital assistant 200 and the wireless communication part 40 is not more than the prescribed distance is performed by, for example, the personal digital assistant 200.

A case will now be described where the determination of whether or not the strength of the radio signal emitted from the wireless communication part 40 and received by the personal digital assistant 200 is not less than the threshold is performed by the toilet device 100 (the controller 30).

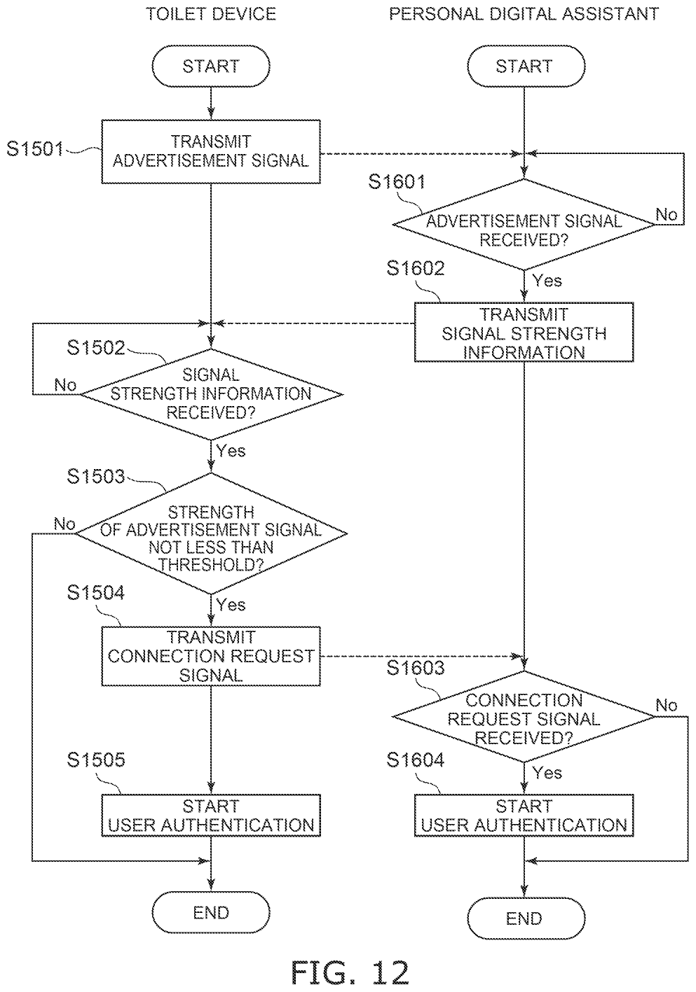

FIG. 12 is a flowchart illustrating an example of an operation in the user authentication of the toilet device and the personal digital assistant according to the embodiment.

As illustrated in FIG. 12, when transmitting the advertisement signal (step S1501), the toilet device 100 (the controller 30) determines whether or not signal strength information is received (step S1502). The signal strength information is related to the strength of the advertisement signal received by the personal digital assistant 200. The toilet device 100 (the controller 30) repeats step S1502 until the signal strength information is received (step S1502: No).

When receiving the signal strength information (step S1502: Yes), the toilet device 100 (the controller 30) determines whether or not the strength of the advertisement signal received by the personal digital assistant 200 is not less than a threshold (step S1503).

When the strength of the advertisement signal received by the personal digital assistant 200 is not less than the threshold (step S1503: Yes), the toilet device 100 (the controller 30) transmits a connection request signal to the personal digital assistant 200 (step S1504) and starts the user authentication (step S1505).

When the strength of the advertisement signal received by the personal digital assistant 200 is less than the threshold (step S1503: No), the toilet device 100 (the controller 30) does not perform step S1504 and step S1505. That is, when the strength of the advertisement signal received by the personal digital assistant 200 is less than the threshold (step S1503: No), the toilet device 100 (the controller 30) does not transmit the connection request signal to the personal digital assistant 200 and does not start the user authentication.

The personal digital assistant 200 determines whether or not the advertisement signal is received (step S1601). The personal digital assistant 200 repeats step S1601 until the advertisement signal is received (step S1601: No).

When the advertisement signal is received (step S1601: Yes), the personal digital assistant 200 transmits the signal strength information to the toilet device 100 (the controller 30) (step S1602).

When transmitting the signal strength information to the toilet device 100 (the controller 30) (step S1602), the personal digital assistant 200 determines whether or not the connection request signal is received (step S1603).

When receiving the connection request signal (step S1603: Yes), the personal digital assistant 200 starts the user authentication (step S1604). When the connection request signal is not received (step S1603: No), the personal digital assistant 200 does not perform step S1604. That is, when the connection request signal is not received (step S1603: No), the personal digital assistant 200 does not start the user authentication.

Thus, the determination of whether or not the strength of the radio signal emitted from the wireless communication part 40 and received by the personal digital assistant 200 is not less than the threshold may be performed by, for example, the toilet device 100 (the controller 30). That is, the determination of whether or not the distance between the personal digital assistant 200 and the wireless communication part 40 is not more than the prescribed distance may be performed by, for example, the toilet device 100 (the controller 30).

The prescribed distance of the determination for the connection of the toilet device 100 (the controller 30) and the personal digital assistant 200 after the user authentication is set to be, for example, greater than the prescribed distance of the determination during the user authentication. That is, the threshold of the strength of the radio signal of the determination for the connection of the toilet device 100 (the controller 30) and the personal digital assistant 200 after the user authentication is set to be, for example, less than the threshold of the strength of the radio signal of the determination during the user authentication. Accordingly, for example, the user can perform the user authentication by passing the personal digital assistant 200 over the toilet device 100 (the casing 14), and then can connect the personal digital assistant 200 to the toilet device 100 while seated on the toilet seat 12. The threshold of the strength of the radio signal of the determination for the connection of the toilet device 100 (the controller 30) and the personal digital assistant 200 after the user authentication may not be set.

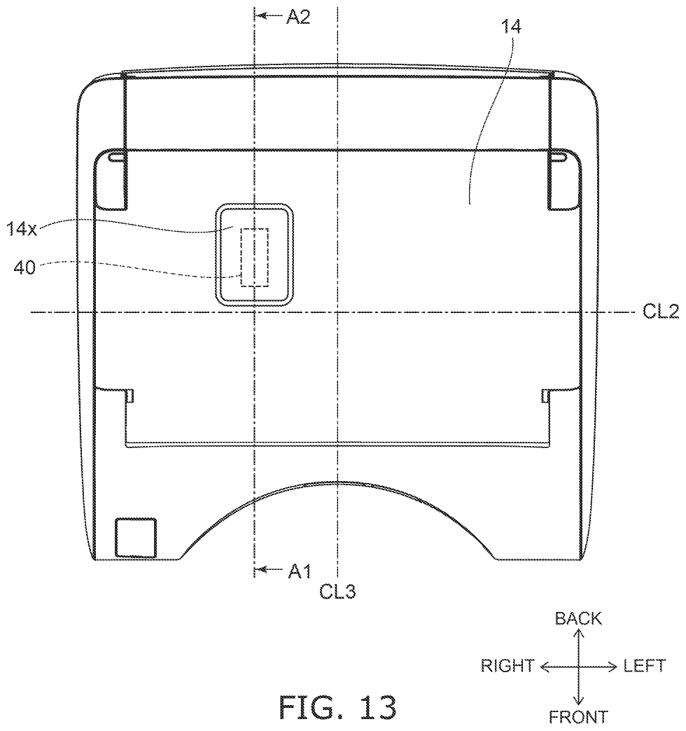

FIG. 13 is a plan view illustrating a part of the toilet device according to the embodiment.

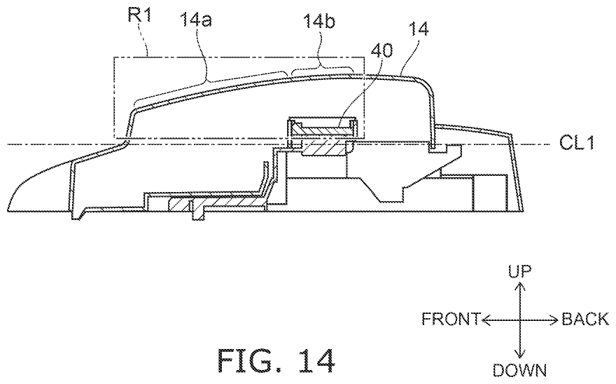

FIG. 14 is a side cross-sectional view illustrating a part of the toilet device according to the embodiment.

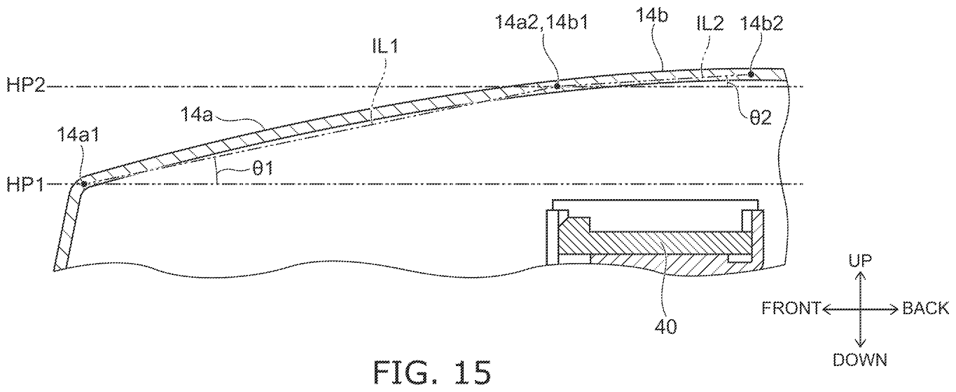

FIG. 15 is a side cross-sectional view illustrating a part of the toilet device according to the embodiment.

FIG. 14 is a cross-sectional view along line A1-A2 shown in FIG. 13.

FIG. 15 is an enlarged view of region R1 shown in FIG. 14.

As illustrated in FIGS. 13 to 15, the wireless communication part 40 is disposed inside the casing 14. More specifically, the casing 14 covers the wireless communication part 40 from above, below, the front, the back, the left, and the right.

For example, at least a part of the wireless communication part 40 is positioned higher than a vertical-direction center CL1 of the casing 14. That is, for example, the upper end of the wireless communication part 40 is positioned higher than the vertical-direction center CL1 of the casing 14. In the example, the entire wireless communication part 40 is positioned higher than the vertical-direction center CL1 of the casing 14. That is, in the example, the lower end of the wireless communication part 40 is positioned higher than the vertical-direction center CL1 of the casing 14. Only a part of the wireless communication part 40 may be positioned higher than the vertical-direction center CL1 of the casing 14. That is, the lower end of the wireless communication part 40 may be positioned lower than the vertical-direction center CL1 of the casing 14.

For example, at least a part of the wireless communication part 40 is positioned behind a longitudinal-direction center CL2 of the casing 14. That is, for example, the back end of the wireless communication part 40 is positioned behind the longitudinal-direction center CL2 of the casing 14. In the example, the entire wireless communication part 40 is positioned behind the longitudinal-direction center CL2 of the casing 14. That is, in the example, the front end of the wireless communication part 40 is positioned behind the longitudinal-direction center CL2 of the casing 14. Only a part of the wireless communication part 40 may be positioned behind the longitudinal-direction center CL2 of the casing 14. That is, the front end of the wireless communication part 40 may be positioned frontward of the longitudinal-direction center CL2 of the casing 14.

For example, the wireless communication part 40 is positioned leftward of a lateral-direction center CL3 of the casing 14 or rightward of the lateral-direction center CL3 of the casing 14. That is, for example, the wireless communication part 40 is disposed at a position at which the wireless communication part 40 does not overlap the lateral-direction center CL3 of the casing 14. In the example, the wireless communication part 40 is positioned rightward of the lateral-direction center CL3 of the casing 14. That is, the left end of the wireless communication part 40 is positioned rightward of the lateral-direction center CL3 of the casing 14. The wireless communication part 40 may be positioned leftward of the lateral-direction center CL3 of the casing 14. That is, the right end of the wireless communication part 40 may be positioned leftward of the lateral-direction center CL3 of the casing 14.

The casing 14 includes a first upper surface part 14a and a second upper surface part 14b. The first upper surface part 14a does not overlap the wireless communication part 40 in the vertical direction. The second upper surface part 14b overlaps the wireless communication part 40 in the vertical direction. The second upper surface part 14b is positioned behind the first upper surface part 14a. For example, the user can perform the user authentication by passing the personal digital assistant 200 over the second upper surface part 14b.

For example, the incline of the second upper surface part 14b is less than the incline of the first upper surface part 14a. In other words, an angle θ2 is, for example, less than an angle θ1, wherein the angle θ1 is the incline angle of the first upper surface part 14a with respect to the horizontal plane, and the angle θ2 is the incline angle of the second upper surface part 14b with respect to the horizontal plane. The angle θ1 is the angle between a horizontal plane HP1 passing through a front end 14a1 of the first upper surface part 14a and a virtual line IL1 connecting the front end 14a1 and a back end 14a2 of the first upper surface part 14a. The angle θ2 is the angle between a horizontal plane HP2 passing through a front end 14b1 of the second upper surface part 14b and a virtual line IL2 connecting the front end 14b1 and a back end 14b2 of the second upper surface part 14b.

The casing 14 also includes an indication part 14x. The indication part 14x is disposed at a position at which the indication part 14x overlaps the wireless communication part 40 in the vertical direction. The indication part 14x may be, for example, an indicator such as a pattern and/or characters printed or engraved in the upper surface of the casing 14, a pattern and/or characters formed by light irradiated on the upper surface of the casing 14, a lamp disposed at the upper surface of the casing 14, etc. For example, the indication part 14x may be a sticker including a pattern and/or characters adhered to the casing 14.

Thus, water contact with the wireless communication part 40 can be suppressed by disposing the wireless communication part 40 inside the casing 14. Also, by disposing the wireless communication part 40 inside the casing 14, for example, compared to when the wireless communication part 40 is disposed at the wall surface WL of the toilet room TR, a reduction of the degree of freedom of the layout inside the toilet room TR can be suppressed.

The detection accuracy of the wireless communication part 40 can be increased by disposing at least a part of the wireless communication part 40 higher than the center CL1 of the casing 14 in the vertical direction.

The wireless communication part 40 can be separated from the opening of the toilet seat 12 by disposing at least a part of the wireless communication part 40 behind the center CL2 of the casing 14 in the longitudinal direction. As a result, for example, when the user authentication is performed by passing the personal digital assistant 200 over the wireless communication part 40, dropping the personal digital assistant 200 through the opening of the toilet seat 12 into the bowl 11a of the toilet 11 can be suppressed.

The wireless communication part 40 can be separated from the opening of the toilet seat 12 by disposing the wireless communication part 40 leftward of the lateral-direction center CL3 of the casing 14 or rightward of the lateral-direction center CL3 of the casing 14. As a result, for example, when performing the user authentication by passing the personal digital assistant 200 over the wireless communication part 40, dropping the personal digital assistant 200 through the opening of the toilet seat 12 into the bowl 11a of the toilet 11 can be suppressed.

The second upper surface part 14b can be a more horizontal surface by setting the incline angle (the angle θ2) with respect to the horizontal plane of the second upper surface part 14b overlapping the wireless communication part 40 in the vertical direction to be less than the incline angle (the angle θ1) with respect to the horizontal plane of the first upper surface part 14a not overlapping the wireless communication part 40 in the vertical direction. As a result, for example, the personal digital assistant 200 does not easily slide frontward when performing the user authentication by passing the personal digital assistant 200 over the wireless communication part 40; and dropping of the personal digital assistant 200 through the opening of the toilet seat 12 into the bowl 11a of the toilet 11 can be suppressed.

The user can more easily ascertain the position of the wireless communication part 40 by disposing the indication part 14x at a position at which the indication part 14x overlaps the wireless communication part 40 in the vertical direction. As a result, for example, the user authentication is easily performed when performing the user authentication by passing the personal digital assistant 200 over the wireless communication part 40.

A case where the proximity sensor 62 is provided as the human body detection sensor 60 will now be described.

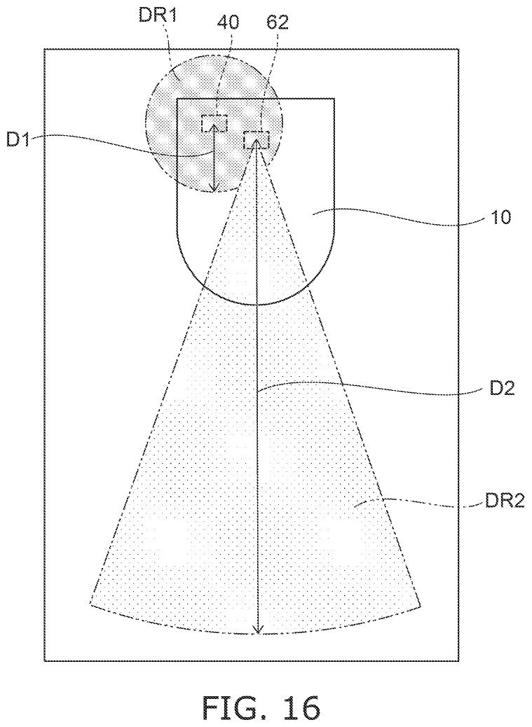

FIG. 16 is a plan view schematically illustrating the toilet device according to the embodiment.

As illustrated in FIG. 16, for example, the toilet device 100 (the controller 30) performs the user authentication when the personal digital assistant 200 is positioned within a detection area DR1 illustrated by a double dot-dash line. A distance D1 from the wireless communication part 40 to the outer perimeter edge of the detection area DR1 is the distance (the prescribed distance) within which the user authentication is performed. The strength of the radio signal emitted from the wireless communication part 40 and received by the personal digital assistant 200 is not less than the threshold when the personal digital assistant 200 is positioned within the detection area DR1.

For example, the proximity sensor 62 detects the user when the user is positioned within a detection area DR2 illustrated by a double dot-dash line. That is, a distance D2 from the proximity sensor 62 to the outer perimeter edge of the detection area DR2 is the distance within which the proximity sensor 62 can detect the user.

For example, it is favorable for the distance D2 within which the human body detection sensor 60 (the proximity sensor 62) can detect the user to be different from the distance D1 (the prescribed distance) within which the user authentication is performed. More specifically, for example, it is favorable for the distance D2 within which the human body detection sensor 60 (the proximity sensor 62) can detect the user to be greater than the distance D1 (the prescribed distance) within which the user authentication is performed. That is, it is favorable for the upper limit of the prescribed distance that is modifiable by the personal digital assistant to be set to be less than the distance D2 within which the human body detection sensor 60 (the proximity sensor 62) can detect the user. For example, it is favorable to limit the value or range of the distance D1 (the prescribed distance) in the personal digital assistant 200 so that the distance D1 (the prescribed distance) can be modified only when different from the distance D2. In other words, for example, it is favorable for the personal digital assistant 200 to display only values or ranges that do not overlap the distance D2 as selectable values or ranges of the distance D1 (the prescribed distance).

Thus, misauthentications can be further suppressed by setting the distance D2 within which the human body detection sensor 60 (the proximity sensor 62) can detect the user to be different from the distance D1 (the prescribed distance) within which the user authentication is performed.

For example, it may be considered to perform the user authentication by passing the personal digital assistant 200 over the casing 14. In such a case, by setting the distance D2 within which the proximity sensor 62 can detect the user to be greater than the prescribed distance D1 within which the user authentication is performed, the proximity sensor 62 can detect the user and automatically open the toilet lid 13 covering the casing 14 before the user authentication is performed. The ease of use can be further improved thereby.

### Method for Controlling Toilet Device

The embodiment also provides a method for controlling the toilet device 100 that can be connected with the personal digital assistant 200 of a user by wireless communication. As illustrated in FIGS. 4 to 12, the method for controlling the toilet device according to the embodiment includes a registration process and a user authentication process. The registration process associates identification information identifying the personal digital assistant 200 and user information identifying the user and stores the identification information and the user information in the storage part 50. The user authentication process is performed after the registration process, and designates the user information and the identification information when the distance between the personal digital assistant 200 and the wireless communication part 40 of the toilet device 100 becomes a prescribed distance or less.

According to the method for controlling the toilet device according to the embodiment, the prescribed distance is modifiable by the personal digital assistant 200.

According to the method for controlling the toilet device according to the embodiment, whether or not the distance between the personal digital assistant 200 and the wireless communication part 40 is not more than the prescribed distance is determined by, for example, whether or not the strength of the radio signal emitted from the wireless communication part 40 and received by the personal digital assistant 200 is not less than a threshold. The threshold is modifiable by the personal digital assistant 200.

### Control Program of Toilet Device

The embodiment also provides a control program installed in the toilet device 100 that can be connected with the personal digital assistant 200 of a user by wireless communication. As illustrated in FIGS. 4 to 12, the control program of the toilet device according to the embodiment causes the toilet device 100 to perform a registration process and a user authentication process. The registration process associates identification information identifying the personal digital assistant 200 and user information identifying the user and stores the identification information and the user information in the storage part 50. The user authentication process is performed after the registration process, and designates the user information and the identification information when the distance between the personal digital assistant 200 and the wireless communication part 40 of the toilet device 100 becomes the prescribed distance or less.

According to the control program of the toilet device according to the embodiment, the prescribed distance is modifiable by the personal digital assistant 200.

According to the control program of the toilet device according to the embodiment, whether or not the distance between the personal digital assistant 200 and the wireless communication part 40 is not more than the prescribed distance is determined by, for example, whether or not the strength of the radio signal emitted from the wireless communication part 40 and received by the personal digital assistant 200 is not less than a threshold. The threshold is modifiable by the personal digital assistant 200.

### Control Program of Personal Digital Assistant

The embodiment also provides a control program installed in a personal digital assistant that can be connected with the toilet device by wireless communication. As illustrated in FIGS. 4 to 12, the control program of the personal digital assistant according to the embodiment causes the personal digital assistant 200 to perform a registration process and a user authentication process. The registration process associates identification information identifying the personal digital assistant 200 and user information identifying the user and stores the identification information and the user information in the storage part 50 of the toilet device 100. The user authentication process is performed after the registration process, and designates the user information and the identification information when the distance between the personal digital assistant 200 and the wireless communication part 40 of the toilet device 100 becomes a prescribed distance or less.

According to the control program of the personal digital assistant according to the embodiment, the prescribed distance is modifiable by the personal digital assistant 200.

According to the control program of the personal digital assistant according to the embodiment, whether or not the distance between the personal digital assistant 200 and the wireless communication part 40 is not more than the prescribed distance is determined by, for example, whether or not the strength of the radio signal emitted from the wireless communication part 40 and received by the personal digital assistant 200 is not less than a threshold. The threshold is modifiable by the personal digital assistant 200.

### Recording Medium

The embodiment also provides a recording medium in which the control program of the toilet device or the control program of the personal digital assistant described above is recorded. The recording medium is at least computer readable. The recording medium may be read-only or enabled for both reading and writing.

Thus, according to the method for controlling the toilet device, the control program of the toilet device, the control program of the personal digital assistant, and the recording medium according to the embodiment, by making the prescribed distance modifiable by the personal digital assistant 200, the distance (the prescribed distance) within which the user authentication is performed can be set to an appropriate distance regardless of the physique of the user and the posture when the user utilizes the toilet device. The user authentication can be more reliably performed thereby.

By determining whether or not the distance between the personal digital assistant 200 and the wireless communication part 40 is not more than the prescribed distance by determining whether or not the strength of the radio signal emitted from the wireless communication part 40 and received by the personal digital assistant 200 is not less than the threshold, and by making the threshold modifiable by the personal digital assistant 200, the radio wave strength (the threshold) at which the user authentication is performed can be set to an appropriate strength regardless of the model of the personal digital assistant 200, the presence and material of a cover of the personal digital assistant 200, etc. As a result, the user authentication not being performed can be suppressed, and misauthentication of a personal digital assistant 200 outside the toilet room TR can be suppressed.

Embodiments may include the following configurations.

### Configuration 1

A toilet device, comprising:
a casing;
a biological information measurement sensor configured to measure biological information of a user;
a controller disposed inside the casing, the controller being configured to transmit the biological information to a personal digital assistant of the user; and
a wireless communication part configured to connect the controller and the personal digital assistant by a wireless communication,
the controller being connected with the personal digital assistant when a distance between the personal digital assistant and the wireless communication part is not more than a prescribed distance,
the wireless communication part being disposed inside the casing.

### Configuration 2

The toilet device according to configuration 1, wherein
at least a part of the wireless communication part is positioned higher than a vertical-direction center of the casing, and
whether or not the distance between the personal digital assistant and the wireless communication part is not more than the prescribed distance is determined by whether or not a strength of a radio signal emitted from the wireless communication part and received by the personal digital assistant is not less than a threshold.

### Configuration 3

The toilet device according to configuration 1 or 2, wherein
at least a part of the wireless communication part is positioned behind a longitudinal-direction center of the casing.

### Configuration 4

The toilet device according to any one of configurations 1 to 3, wherein
the wireless communication part is positioned
   leftward of a lateral-direction center of the casing, or
   rightward of the lateral-direction center of the casing.

### Configuration 5

The toilet device according to configuration 3, wherein
the casing includes:
   a first upper surface part not overlapping the wireless communication part in the vertical direction; and
   a second upper surface part positioned behind the first upper surface part, the second upper surface part overlapping the wireless communication part in the vertical direction, and
an incline angle of the second upper surface part with respect to a horizontal plane is less than an incline angle of the first upper surface part with respect to the horizontal plane.

### Configuration 6

The toilet device according to any one of configurations 1 to 5, wherein
the casing includes an indication part disposed at a position at which the indication part overlaps the wireless communication part in the vertical direction.

### Configuration 7

The toilet device according to any one of configurations 1 to 6, further comprising:
a storage part,
the controller being configured to perform a registration mode including
   associating identification information identifying the personal digital assistant and user information identifying the user, and
   storing the identification information and the user information in the storage part,
after performing the registration mode, the controller performing a user authentication of designating the user information and the identification information when the distance between the personal digital assistant and the wireless communication part becomes the prescribed distance or less,
the prescribed distance being modifiable by the personal digital assistant.

### Configuration 8

The toilet device according to configuration 7, wherein
whether or not the distance between the personal digital assistant and the wireless communication part is not more than the prescribed distance is determined by whether or not a strength of a radio signal emitted from the wireless communication part and received by the personal digital assistant is not less than a threshold, and
the threshold is modifiable by the personal digital assistant.

### Configuration 9

The toilet device according to configuration 7 or 8, further comprising:
a human body detection sensor configured to detect the user,
the controller performing the user authentication when the human body detection sensor detects the user and the distance between the personal digital assistant and the wireless communication part is not more than the prescribed distance.

### Configuration 10

The toilet device according to configuration 9, wherein
the human body detection sensor includes a proximity sensor configured to detect an approach of the user, and
a distance at which the proximity sensor can detect the user is different from the prescribed distance.

### Configuration 11

A method for controlling a toilet device, the toilet device being connectable with a personal digital assistant of a user by a wireless communication, the method comprising:
a registration process including
   associating identification information identifying the personal digital assistant and user information identifying the user, and
   storing the identification information and the user information in a storage part; and
a user authentication process performed after the registration process, the user authentication process designating the user information and the identification information when a distance between the personal digital assistant and a wireless communication part of the toilet device becomes a prescribed distance or less,
the prescribed distance being modifiable by the personal digital assistant.

### Configuration 12

A recording medium, a control program being recorded in the recording medium, the control program being installed in a toilet device, the toilet device being connectable with a personal digital assistant of a user by a wireless communication,
the control program being configured to cause the toilet device to perform:
   a registration process including
      associating identification information identifying the personal digital assistant and user information identifying the user, and
      storing the identification information and the user information in a storage part; and
   a user authentication process performed after the registration process, the user authentication process designating the user information and the identification information when a distance between the personal digital assistant and a wireless communication part of the toilet device becomes a prescribed distance or less,
the prescribed distance being modifiable by the personal digital assistant.

Thus, according to embodiments, a toilet device is provided in which water contact with the wireless communication part can be suppressed, and a reduction of the degree of freedom of the layout inside the toilet room can be suppressed.

According to embodiments, a toilet device, a method for controlling a toilet device, and a recording medium are provided in which the user authentication can be more reliably performed.

The invention has been described with reference to the embodiments. However, the invention is not limited to these embodiments. Any design changes in the above embodiments suitably made by those skilled in the art are also encompassed within the scope of the invention as long as they fall within the spirit of the invention. For example, the shape, the size the material, the disposition and the arrangement or the like of the components included in the toilet device are not limited to illustrations and can be changed appropriately.

The components included in the embodiments described above can be combined to the extent possible, and these combinations are also encompassed within the scope of the invention as long as they include the features of the invention.

What is claimed is:

1. A toilet device, comprising:
a casing;
a biological information measurement sensor configured to measure biological information of a user;
a controller disposed inside the casing, the controller being configured to transmit the biological information to a personal digital assistant of the user; and
a wireless communication part configured to connect the controller and the personal digital assistant by a wireless communication,
the controller being connected with the personal digital assistant when a distance between the personal digital assistant and the wireless communication part is not more than a prescribed distance,
the wireless communication part being disposed inside the casing.

2. The toilet device according to claim 1, wherein
at least a part of the wireless communication part is positioned higher than a vertical-direction center of the casing, and
whether or not the distance between the personal digital assistant and the wireless communication part is not more than the prescribed distance is determined by whether or not a strength of a radio signal emitted from the wireless communication part and received by the personal digital assistant is not less than a threshold.

3. The toilet device according to claim 2, wherein
at least a part of the wireless communication part is positioned behind a longitudinal-direction center of the casing.

4. The toilet device according to claim 2, wherein
the wireless communication part is positioned
leftward of a lateral-direction center of the casing, or
rightward of the lateral-direction center of the casing.

5. The toilet device according to claim 3, wherein
the casing includes:
a first upper surface part not overlapping the wireless communication part in the vertical direction; and
a second upper surface part positioned behind the first upper surface part, the second upper surface part overlapping the wireless communication part in the vertical direction, and
an incline angle of the second upper surface part with respect to a horizontal plane is less than an incline angle of the first upper surface part with respect to the horizontal plane.

6. The toilet device according to claim 2, wherein
the casing includes an indication part disposed at a position at which the indication part overlaps the wireless communication part in the vertical direction.

7. The toilet device according to claim 1, further comprising:
a storage part,
the controller being configured to perform a registration mode including
associating identification information identifying the personal digital assistant and user information identifying the user, and
storing the identification information and the user information in the storage part,
after performing the registration mode, the controller performing a user authentication of designating the user information and the identification information when the distance between the personal digital assistant and the wireless communication part becomes the prescribed distance or less,
the prescribed distance being modifiable by the personal digital assistant.

8. The toilet device according to claim 7, wherein
whether or not the distance between the personal digital assistant and the wireless communication part is not more than the prescribed distance is determined by whether or not a strength of a radio signal emitted from the wireless communication part and received by the personal digital assistant is not less than a threshold, and
the threshold is modifiable by the personal digital assistant.

9. The toilet device according to claim 7, further comprising:
a human body detection sensor configured to detect the user,
the controller performing the user authentication when the human body detection sensor detects the user and the distance between the personal digital assistant and the wireless communication part is not more than the prescribed distance.

10. The toilet device according to claim 9, wherein
the human body detection sensor includes a proximity sensor configured to detect an approach of the user, and
a distance at which the proximity sensor can detect the user is different from the prescribed distance.

11. A method for controlling a toilet device, the toilet device being connectable with a personal digital assistant of a user by a wireless communication, the method comprising:
a registration process including
associating identification information identifying the personal digital assistant and user information identifying the user, and
storing the identification information and the user information in a storage part; and
a user authentication process performed after the registration process, the user authentication process designating the user information and the identification information when a distance between the personal digital assistant and a wireless communication part of the toilet device becomes a prescribed distance or less,
the prescribed distance being modifiable by the personal digital assistant.

12. A recording medium, a control program being recorded in the recording medium, the control program being installed in a toilet device, the toilet device being connectable with a personal digital assistant of a user by a wireless communication,
the control program being configured to cause the toilet device to perform:
a registration process including
associating identification information identifying the personal digital assistant and user information identifying the user, and
storing the identification information and the user information in a storage part; and
a user authentication process performed after the registration process, the user authentication process designating the user information and the identification information when a distance between the personal digital assistant and a wireless communication part of the toilet device becomes a prescribed distance or less,
the prescribed distance being modifiable by the personal digital assistant.
